(19) United States
(12) Patent Application Publication
(10) Pub. No.: US 2018/0140451 A1
(43) Pub. Date: May 24, 2018

(54) Ankle Brace with Enhanced Lacing

(71) Applicant: Medical Specialties, Inc., Charlotte, NC (US)

(72) Inventor: Eric Lee Gaylord, Matthews, NC (US)

(21) Appl. No.: 15/816,469

(22) Filed: Nov. 17, 2017

Related U.S. Application Data
(60) Provisional application No. 62/424,116, filed on Nov. 18, 2016.

(51) Int. Cl. A61F 5/01

(52) U.S. Cl. CPC A61F 5/0111

(57) ABSTRACT

The present ankle brace includes an advantageous lacing configuration that helps provide cast-like compression on a patient's lower leg, ankle, and foot. Pulling the lacing mechanism equilibrates lace tension along the length of the ankle brace in a way that applies consistent compression to the patient's lower leg and ankle.

### CROSS-REFERENCE TO PRIORITY APPLICATION

This application hereby claims the benefit of pending U.S. Patent Application No. 62/424,116 for an Ankle Brace with Enhanced Lacing (filed Nov. 18, 2016), which is hereby incorporated by reference in its entirety.

### FIELD OF THE INVENTION

The present invention relates to an ankle brace for immobilizing and protecting a patient's lower leg, ankle, and foot.

### BACKGROUND

Immobilizing a patient's lower leg, ankle, and foot can promote healing of bone fractures and soft-tissue injuries (e.g., sprains), and so ankle braces and supports are often used for this purpose. Moreover, athletes often use ankle braces to prevent such injuries. Ankles are especially vulnerable to sprains, strains, and fractures. As is known to athletes, the ankle is often injured by contact with other athletes or sports equipment, or by the ankle assuming an unnatural position during sports and exercise. Injuries often occur during movements associated with athletics, such as running, jumping, and falling.

Many ankle injuries occur either when the foot rolls or falls inwardly (referred to as “inversion”) so that the outer (i.e., lateral) ligaments become overstretched, or when the foot rolls or falls outwardly (referred to as “eversion”) so that the inner ligament (i.e., the deltoid ligament) becomes overstretched. Many athletes rely upon taping to provide supplemental ankle support, whereby the athlete or trainer winds athletic tape around the athlete's ankle to limit the motion of the ankle relative to the leg. Although taping stabilizes the ankle against undesired motion, several drawbacks exist. For example, taping may restrict all motion of the ankle, whether desirable or undesirable. The restrictive characteristics of taping, especially with respect to plantar flexion and dorsiflexion, may hinder the athlete's ability to perform. Further, tape tends to stretch and loosen as the athlete moves, thereby decreasing its effectiveness in supporting the ankle.

Ankle-stabilizing devices include boot-shaped members that cover the athlete's foot and ankle, and supplemental straps designed to wrap around and stabilize certain areas of the athlete's foot and ankle. Some ankle-stabilizing devices include lace-eyelet fasteners to secure the athlete's foot and ankle. The laces are typically threaded through the eyelets and the device may be tightened around the foot and ankle by directly pulling and tying the laces.

### SUMMARY

The present ankle brace includes an advantageous lacing configuration that helps provide cast-like compression on a patient's lower leg, ankle, and foot to restrict flexion, extension, and lateral movement of the ankle joint. By pulling an improved lacing mechanism, tension in a lace equilibrates along the length of the ankle brace in a way that applies consistent compression to the patient's lower leg and ankle.

Exemplary ankle braces (e.g., ankle-brace splints) include an ankle support sleeve and a connecting front-panel tongue (e.g., a stretchable tongue) that together form an ankle-brace boot, which is securely positioned around a patient's ankle and lower leg. A lace, which typically resists excessive stretching, is threaded through lace-redirection mechanisms (e.g., eyelets) positioned on the ankle support sleeve (e.g., along the ankle support sleeve's free front edges) and through a lacing closure tab. Regardless of the shape and girth of the patient's lower leg, pulling the lacing closure tab circumferentially around the ankle-brace boot and then releasably securing the lacing closure tab to the ankle-brace boot helps achieve cast-like compression of the ankle brace to the patient's lower leg and ankle.

The foregoing illustrative summary, as well as other exemplary objectives and/or advantages of the invention, and the manner in which the same are accomplished, are further explained within the following detailed description and its accompanying drawings and photographs.

### BRIEF DESCRIPTION OF THE DRAWINGS

FIGS. 1-4 depict an exemplary ankle-brace embodiment having an advantageous lacing configuration.

FIGS. 5-6 depict another exemplary ankle-brace embodiment having an advantageous lacing configuration.

### DETAILED DESCRIPTION

The present ankle braces with improved lacing are described herein with reference to the accompanying drawings, which depict exemplary ankle-brace embodiments that should not be construed as limiting the scope of the invention. Rather, the exemplary ankle-brace embodiments are provided so this disclosure will be thorough and complete to convey to those having ordinary skill in the art the scope of the invention. In the accompanying drawings, like numbers refer to like elements.

As described herein, the terms “interior surface” and “inner surface” refer to a two-dimensional surface or side closest to the patient's ankle, and the terms “exterior surface” and “outer surface” refer to a two-dimensional surface or side farthest from the patient's ankle (i.e., an outer surface is opposite an inner surface).

The term “section” refers in context to a portion or an area of an ankle support sleeve (e.g., an ankle-brace sleeve). The term “proximal” refers to the part of the ankle brace or lower leg that is closer to the patient's knee, and the term “distal” refers to the part of the ankle brace or lower leg farther from the patient's knee.

The terms “positioned” and “positioning” are used conventionally to refer to one element being located relative to another element. This embraces one element being fixed or releasably secured to another element. For example, a first element may be positioned against a second element (or positioned between second and third elements) by sewing, by hook-and-loop fasteners, or by other known mechanisms for physically attaching physical elements to each other. The terms “fixed,” “affixed,” and “secured” may include sewn, made integral with, adhered with adhesive, bonded (e.g., fused with heat), or otherwise attached in any suitable manner.

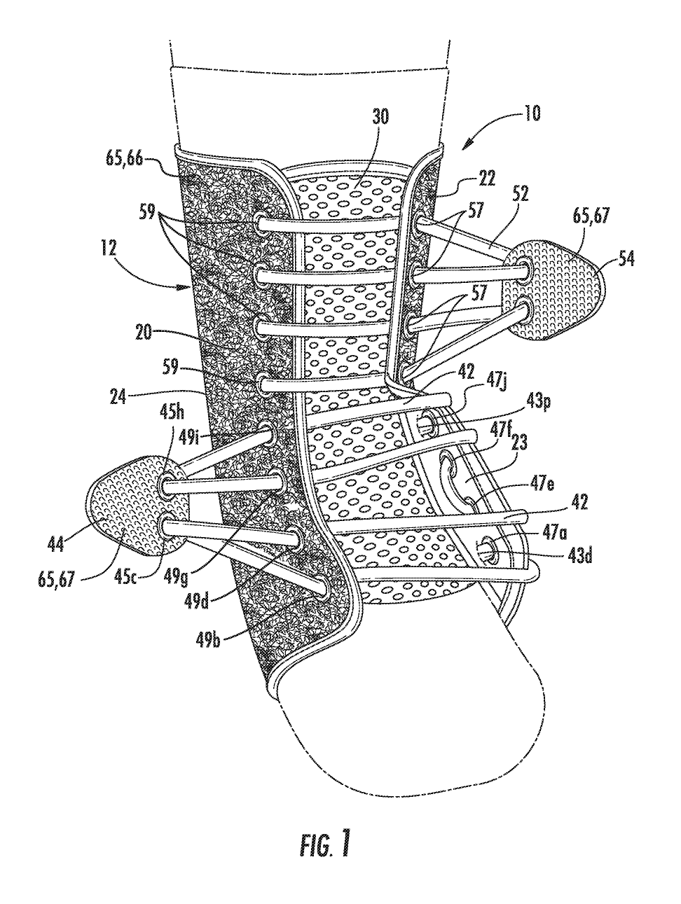

The terms “freely threaded” and “freely interlaced” are used herein in accordance with the ankle-brace embodiments depicted in FIGS. 1-6 to refer to a lace that is moveably connected to a lace-redirection mechanism, such as an eyelet, rather than fixedly attached to a lace-redirection mechanism. For example, as described herein, a lace that is “freely threaded” through a closure-tab lace-redirection mechanism, which is positioned on or otherwise formed through a lacing closure tab, can facilitate movement of the lacing closure tab along the lace.

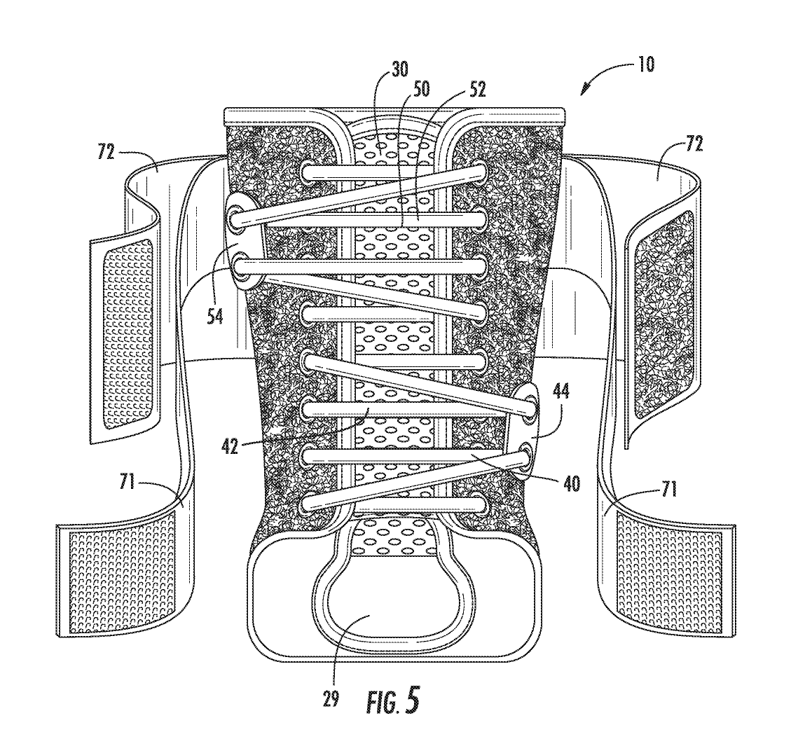

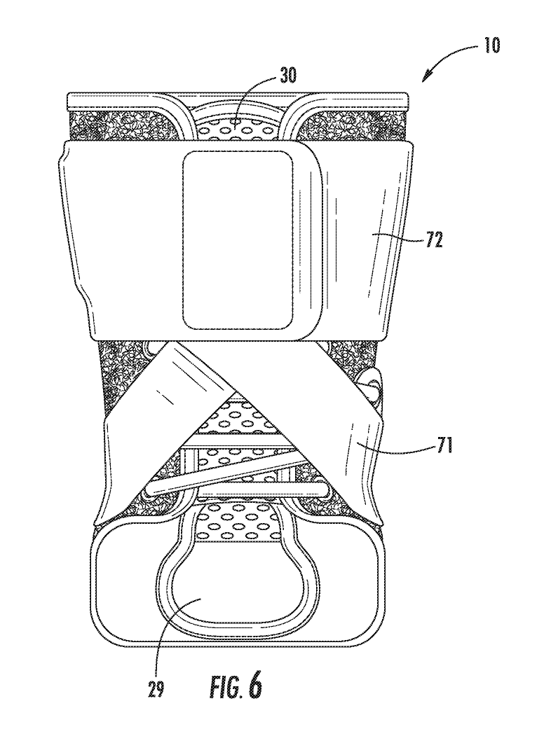

FIGS. 1-4 and FIGS. 5-6 depict exemplary ankle-brace embodiments sized for a patient. These exemplary ankle braces, which can be worn with or without athletic socks (and within or without a shoe), are typically configured to fit both left and right ankles. To highlight the advantageous lacing configuration according to the present invention, the ankle-brace embodiment depicted in FIGS. 1-4 omits both the optional stabilizing straps and the optional binding straps. Appendix 1 includes photographs of an exemplary prototype of the ankle brace depicted in FIGS. 5-6. The description of the abridged ankle-brace embodiment depicted in FIGS. 1-4 typically applies to the ankle-brace embodiment depicted in FIGS. 5-6, except as noted in the supporting description, figures, and photographs. As noted, this U.S. nonprovisional application incorporates by reference priority U.S. Patent Application Ser. No. 62/424,116, including its photographs provided as Appendix I.

With reference to the exemplary ankle brace 10 depicted in FIGS. 1-4, the ankle brace 10 includes an ankle-brace boot 12 defining an interior surface and an exterior surface. The ankle-brace boot 12, which is configured to receive a foot and an ankle, is partly formed by an ankle support sleeve 20, which includes at least a first side-sleeve section 22 and a second side-sleeve section 24. The first side-sleeve section 22 and the second side-sleeve section 24 represent opposite sides of the ankle brace 10. For example, if the first side-sleeve section 22 is a left quarter panel of the ankle-brace boot 12, the second side-sleeve section 24 is a right quarter panel of the ankle-brace boot 12, and vice-versa. The first side-sleeve section 22 and the second side-sleeve section 24 typically define respective free front edges toward the front of the ankle support sleeve 20.

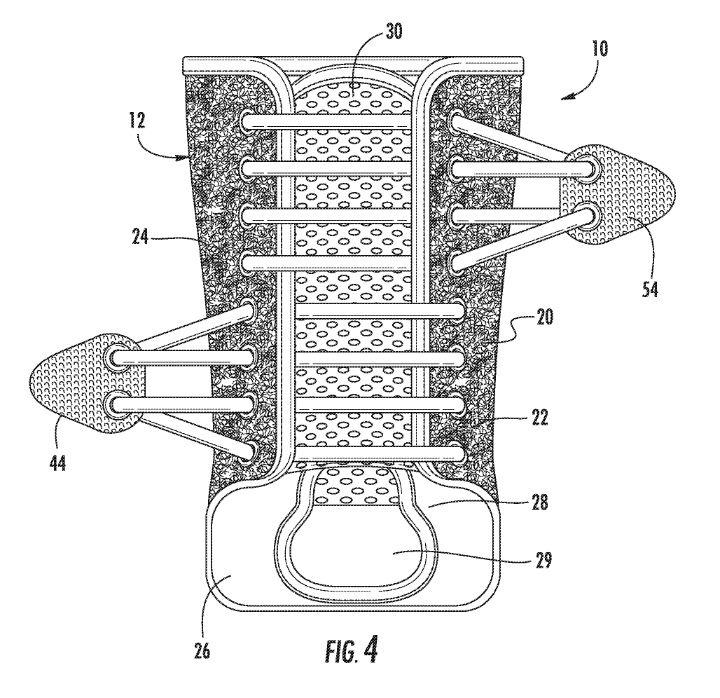

The ankle support sleeve 20 may include an optional bottom-sleeve section 26 (FIG. 4) positioned beneath and between, and connected to, the first side-sleeve section 22 and the second side-sleeve section 24. When the ankle brace 10 is secured to a patient's ankle, the patient's rear sole should be placed against the bottom-sleeve section 26. Similarly, the ankle support sleeve 20 may include an optional rear-sleeve section 28 (FIG. 4) positioned between and connected to both the first side-sleeve section 22 and the second side-sleeve section 24. When the ankle brace 10 is secured to a patient's ankle, the patient's Achilles tendon and lower leg should be placed against the rear-sleeve section 28. The rear-sleeve section 28 may include cushioning to provide the patient with enhanced comfort and support. More generally, padding may be applied to the interior surfaces of the ankle-brace boot 12 to help engage the patient's ankle and to improve patient comfort.

In exemplary ankle-brace embodiments, the ankle support sleeve 20 is a boot-like body member that is substantially L-shaped to cover at least a lower and rear portion of the patient's foot and ankle. In these exemplary ankle-brace embodiments, ankle support sleeve 20 includes a stirrup-like portion (e.g., the bottom-sleeve section 26) that conforms to the lower surfaces of the foot by extending under portions of the patient's calcaneus. In these exemplary ankle-brace embodiments as depicted in FIG. 4, the ankle support sleeve 20 typically defines a heel opening 29 (e.g., for receiving a portion of the patient's heel).

In other exemplary ankle-brace embodiments, the ankle support sleeve 20 excludes a stirrup-like portion (e.g., excludes the bottom-sleeve section 26) such that ankle support sleeve 20 has a lower edge ending approximately below the malleoli (e.g., immediately below the malleoli). In these exemplary ankle-brace embodiments, the ankle support sleeve 20 does not extend under the patient's foot and so is more U-shaped (or C-shaped) than L-shaped.

Typically, the ankle support sleeve 20 is substantially inelastic (e.g., formed from flexible, low-stretch material) to help stabilize the patient's ankle and lower leg. In another exemplary embodiment, the rear-sleeve section 28 includes heel cushioning (e.g., via elastic material, such as stretchable, mesh fabric) positioned above the heel opening 29. The ankle support sleeve 20 may be constructed of substantially inelastic fabric material that resists stretching and elongation. In an exemplary ankle-brace embodiment, the substantially inelastic material is lightweight nylon having excellent strength and durability (e.g., woven ballistic nylon fabric). As used herein, the term “elastic” generally refers to material that can be readily stretched or expanded, and then can return to its initial shape (i.e., elastic materials resist permanent deformation by stretching), and the term “inelastic” generally refers to material that resists stretching and elongation.

In the exemplary ankle-brace embodiments depicted in FIGS. 1-6, the ankle-brace boot 12 includes a tongue 30 (e.g., a front-panel tongue), which is typically connected to the ankle support sleeve's first side-sleeve section 22 and the ankle support sleeve's second side-sleeve section 24. The tongue 30, which is usually positioned between the first side-sleeve section's free front edge and the second side-sleeve section's free front edge, is typically affixed to the ankle support sleeve 20. For example, the tongue 30 may be secured (e.g., tacked or stitched) to the ankle support sleeve 20 only at the respective distal ends of the first side-sleeve section 22 and the second side-sleeve section 24. Typically, the tongue 30 is stretchable to facilitate placement of the ankle brace 10 on a patient's ankle and cushioned for the patient's comfort. For example, the tongue 30 may be formed of stretchable, mesh fabric. The tongue 30 facilitates placement of the ankle-brace boot 12 to the patient's foot and ankle, and provides padding between the ankle-brace laces and the patient's foot and ankle.

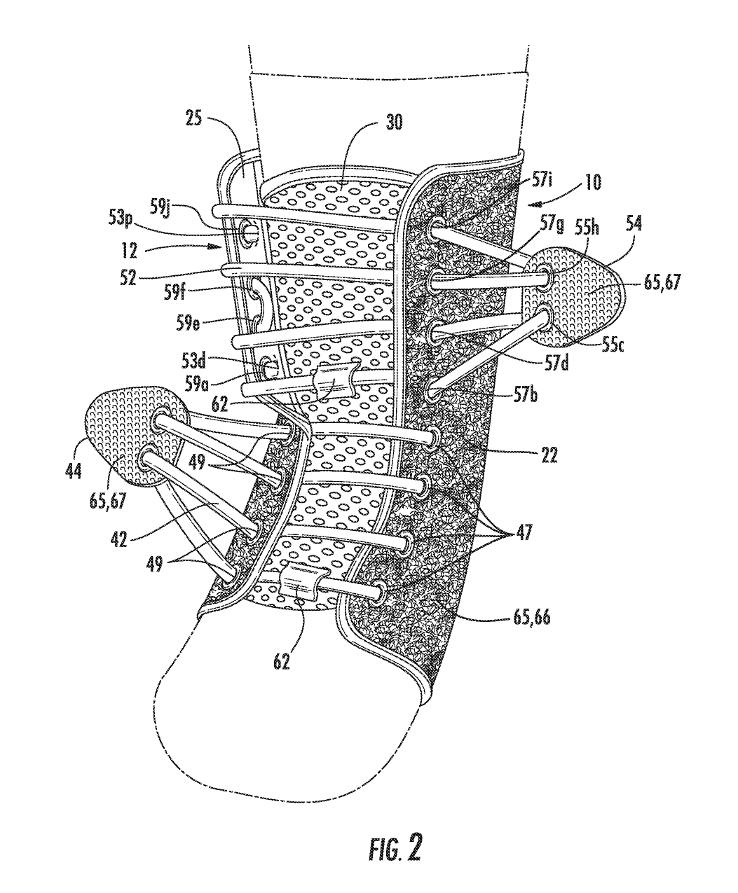

Together, the ankle support sleeve 20 and the tongue 30 substantially form the ankle-brace boot 12. For example, the tongue 30 closes the gap (e.g., the throat) in the ankle support sleeve 20, thereby forming the ankle-brace boot 12. The tongue 30 is typically positioned within the gap or throat in the ankle support sleeve 20 so as to extend along both a first outer side-sleeve strip 23 (FIG. 1) and second outer side-sleeve strip 25 (FIG. 2). The first outer side-sleeve strip 23, which is typically a marginal portion of the first side-sleeve section 22, may extend from proximate the tongue 30 to an adjacent outer edge of the first side-sleeve section 22. The second outer side-sleeve strip 25, which is typically a marginal portion of the second side-sleeve section 24, may extend from proximate the tongue 30 to an adjacent outer edge of the second side-sleeve section 24. Accordingly, the first outer side-sleeve strip 23 may be partly bounded by the tongue 30 and an adjacent outer edge of the first side-sleeve section 22, and the second outer side-sleeve strip 25 may be partly bounded by the tongue 30 and an adjacent outer edge of the second side-sleeve section 24.

Eyelets (or similar holes or openings, such as reinforced holes and/or slots, or other lace-redirection mechanisms, such as loops, hooks, folded webbing, and/or buckles), are typically formed through or otherwise positioned at or near (e.g., directly or indirectly connected to or otherwise positioned upon) both the first side-sleeve section 22 and the second side-sleeve section 24. In an exemplary ankle-brace embodiment, a first series of lace-redirection mechanisms are positioned at or near the ankle support sleeve's first side-sleeve section 22 (e.g., a first series of eyelets formed at a free front edge near the tongue 30), and a second series of lace-redirection mechanisms are positioned at or near the ankle support sleeve's second side-sleeve section 24 (e.g., a second series of eyelets formed at a free front edge near the tongue 30). One or more laces are then freely threaded (or otherwise freely interlaced) through these respective lace-redirection mechanisms (e.g., the laces are not fixedly attached to the respective lace-redirection mechanisms and can move through or along the lace-redirection mechanisms). In this regard, the first outer side-sleeve strip 23 and the second outer side-sleeve strip 25 can function as eyestays for the ankle brace 10. For example, first and second outer side-sleeve strips 23, 25 may be reinforced marginal portions of the first and second side-sleeve sections 22, 24, respectively. Those having ordinary skill in the art will understand that exemplary ankle braces according to the present invention may employ any lace-redirection mechanisms (e.g., reinforced holes and slots, or external loops, hooks, folded webbing, and/or buckles) in addition to or instead of eyelets.

Moreover, it is within the scope of the present ankle-brace invention to include one or more external flaps (not shown) on the exterior of the ankle support sleeve to facilitate redirection of a lace. In one ankle-brace embodiment, a first flap having a series of eyelets or other lace-redirection mechanisms can be positioned upon the ankle support sleeve's first side-sleeve section (e.g., near the tongue). A lace may be guided through the first flap's lace-redirection mechanisms (e.g., eyelets) and a series of eyelets or other lace-redirection mechanisms positioned upon the ankle support sleeve's second side-sleeve section to promote even closure of the ankle-brace boot to help achieve cast-like compression on a patient's lower leg, ankle, and foot. In another ankle-brace embodiment, a second flap having a series of eyelets or other lace-redirection mechanisms can be positioned upon the ankle support sleeve's second side-sleeve section (e.g., near the tongue). A lace may be guided through the second flap's lace-redirection mechanisms (e.g., eyelets) and a series of eyelets or other lace-redirection mechanisms positioned upon the ankle support sleeve's first side-sleeve section to promote even closure of the ankle-brace boot to help achieve cast-like compression on a patient's lower leg, ankle, and foot. In yet another ankle-brace embodiment, a lace may be guided through both (i) the first flap having eyelets or other lace-redirection mechanisms and (ii) the second flap having eyelets or other lace-redirection mechanisms to help achieve cast-like compression on a patient's lower leg, ankle, and foot, thereby restricting flexion, extension, and lateral movement of the ankle joint. Such first and second flaps can indirectly connect the lace-redirection mechanisms to the respective first and second side-sleeve sections.

The ankle support sleeve 20 may be formed from one or more sheets of fabric. In one embodiment, the ankle support sleeve 20 is a “one-piece” body member formed from a single sheet of material that is capable of forming an L-shaped, boot-like shape (e.g., the ankle-brace boot 12 without the tongue 30). In another embodiment, the ankle support sleeve 20 is a “multi-piece” body member formed from two or more sheets or strips of material to form an L-shaped, boot-like shape (e.g., the ankle-brace boot 12 without the tongue 30).

Exemplary ankle braces may employ one or more lacing interconnection systems. A typical lacing interconnection system according to the present invention includes (i) lace-redirection mechanisms positioned on the ankle-brace boot (e.g., respective series of eyelets positioned at the ankle support sleeve's first side-sleeve section and second side-sleeve section), (ii) a lacing closure tab (e.g., including one or more closure-tab eyelets or other lace-redirection mechanisms, such as reinforced holes and/or slots, or external loops, hooks, folded webbing, and/or buckles) and (iii) a lace attached to and connecting the ankle-brace boot and the lacing closure tab. When secured to the patient's ankle via the lacing interconnection system, an exemplary ankle brace restricts flexion, extension, and lateral movement of the patient's ankle joint to help provide cast-like compression of the ankle brace to a patient's lower leg, ankle, and foot.

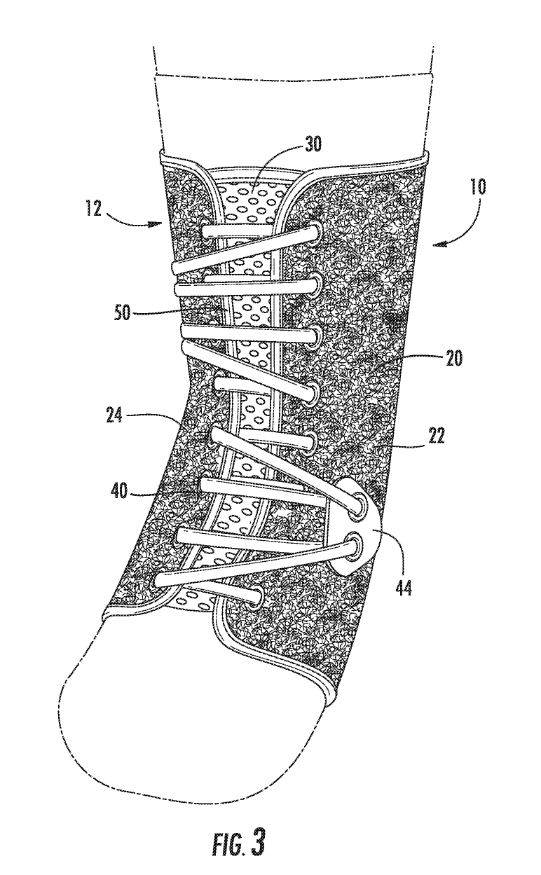

The exemplary ankle-brace embodiment depicted in FIGS. 1-4 includes two lacing interconnection systems, namely a lower lacing interconnection system 40, which is nearer the patient's toe when the ankle brace is worn, and an upper lacing interconnection system 50, which is nearer the patient's knee when the ankle brace is worn. As viewed from the patient's perspective and as depicted in FIG. 3, the lower lacing interconnection system 40 closes from right to left, and the upper lacing interconnection system 50 closes from left to right. As viewed from the patient's perspective (and to describe representative ankle-brace embodiments), the ankle support sleeve's first side-sleeve section 22 is positioned to the left, and the ankle support sleeve's second side-sleeve section 24 is positioned to the right. Although such a reciprocal configuration of the lacing interconnection systems 40, 50 is typical, alternative ankle-brace embodiments may employ parallel configurations, whereby both the lower lacing interconnection system 40 and the upper lacing interconnection system 50 close from right to left (or from left to right). As noted, exemplary ankle braces can be configured to fit both left and right ankles, so the references to first and second sides (e.g., left and right from the patient's perspective) are for purposes of description and not for limitation.

The exemplary lower lacing interconnection system 40 depicted in FIGS. 1-4 includes a lower lace 42 (e.g., exactly one lace) that moveably connects a lower lacing closure tab 44 (via one or more lace-redirection mechanisms, such as lower closure tab eyelets 45) to lower lace-redirection mechanisms (e.g., lower eyelets 47, 49) positioned on the ankle support sleeve 20. Those having ordinary skill in the art will appreciate that the lower lacing closure tab 44 is moveably connected to the lower lace 42. When the lower lacing closure tab 44 is not releasably secured to another part of the ankle brace (e.g., releasably affixed to the ankle support sleeve 20), the lower lacing closure tab 44 can move substantially freely along the lower lace 42 (e.g., the lower lace 42 can pass through the lower closure tab eyelets 45).

With reference to the exemplary ankle-brace embodiment depicted in FIGS. 1-4, four lower first side-sleeve eyelets 47 are formed through or otherwise positioned upon the ankle support sleeve's first side-sleeve section 22 near its free front edge (e.g., four lower eyelets are positioned in the first outer side-sleeve strip 23), and lower second side-sleeve eyelets 49 are formed through or otherwise positioned upon the ankle support sleeve's second side-sleeve section 24 near its free front edge (e.g., four lower eyelets are positioned in the second outer side-sleeve strip 25).

The lower lacing closure tab 44 includes a releasable-attachment mechanism, such as a hook-and-loop surface, to releasably secure the lower lacing closure tab 44 to another part of the ankle brace 10. As illustrated in FIGS. 1-4, two lower closure-tab eyelets 45c, 45h are formed through or otherwise positioned upon the lower lacing closure tab 44. The lower closure-tab eyelets 45 help to ensure that the lower lacing closure tab 44 remains centered on the ankle support sleeve 20 as the lower lacing closure tab 44 is pulled over and around the ankle-brace boot 12.

The lower lace 42 is freely threaded (or otherwise interlaced) though the lower first side-sleeve eyelets 47, the lower second side-sleeve eyelets 49, and the lower closure-tab eyelets 45, respectively. The lower lace 42 can move freely to accommodate the typical non-cylindrical anatomy of a patient's lower leg in which the leg tapers from the calf to the ankle (e.g., a frustoconical shape). This free movement of the lower lace 42 facilitates consistent tension along its length. This even tension is transferred to the lower first side-sleeve eyelets 47, the lower second side-sleeve eyelets 49, and the lower closure-tab eyelets 45 through which the lower lace 42 is interlaced. The lower lace 42 and the moveably attached lower lacing closure tab 44 wrap circumferentially around—rather than helically along—the patient's lower leg and ankle in a way that uniformly closes and tensions the ankle-brace boot 12.

Typically, a lower lace 42 has (i) a first end portion secured (e.g., affixed) to the ankle support sleeve 20 near the ankle support sleeve's distal end (e.g., secured at either the distal end of the first outer side-sleeve strip 23 or the distal end of the second outer side-sleeve strip 25) and (ii) a second end portion secured (e.g., affixed) to the ankle support sleeve 20 approximately midway along the ankle support sleeve 20 (e.g., secured midway along the ankle support sleeve 20 toward the proximal end of the first outer side-sleeve strip 23 or the proximal end of the second outer side-sleeve strip 25).

In the exemplary ankle-brace embodiment depicted in FIGS. 1-4, one lower lace 42 has (i) a first distal end fixed to the ankle support sleeve's interior surface nearer the ankle support sleeve's distal end (i.e., a lower distal fixation 43d toward the toe-side end of the first outer side-sleeve strip 23) and (ii) a second proximal end fixed to the ankle support sleeve's interior surface nearer the ankle support sleeve's proximal end (i.e., a lower proximal fixation 43p secured midway along the ankle support sleeve 20 toward the knee-side end of the first outer side-sleeve strip 23). In the exemplary ankle-brace embodiment depicted in FIGS. 1-4, four lower first side-sleeve eyelets 47a, 47e, 47f, 47j are positioned at the ankle support sleeve's first side-sleeve section 22 between the lower lace's lower distal fixation 43d and the lower lace's lower proximal fixation 43p, and four lower second side-sleeve eyelets 49b, 49d, 49g, 49i are positioned at the ankle support sleeve's second side-sleeve section 24. As noted, the first outer side-sleeve strip 23 and the second outer side-sleeve strip 25 can function as eyestays (e.g., the four lower first side-sleeve eyelets 47a, 47e, 47f, 47j can be formed through the first outer side-sleeve strip 23, and the four lower second side-sleeve eyelets 49b, 49d, 49g, 49i can be formed through second outer side-sleeve strip 25).

As illustrated in FIGS. 1-4 in which the ends of lower lace 42 are fixed to the interior surface of the first side-sleeve section 22 (e.g., at the first outer side-sleeve strip 23 on the left side as viewed from the patient's perspective), pulling the lower lacing closure tab 44 away from the ankle support sleeve's second side-sleeve section 24 (and over and around the ankle support sleeve's first side-sleeve section 22) uniformly closes and tensions the ankle-brace boot 12 by drawing together the ankle support sleeve's second side-sleeve section 24 and ankle support sleeve's first side-sleeve section 22. As viewed from the patient's perspective, the lower lacing interconnection system 40 closes from right to left as shown in the exemplary ankle brace of FIGS. 1-4.

As illustrated in the exemplary ankle-brace embodiment depicted in FIGS. 1-4, the ankle brace 10 includes a compression-enhancing lacing configuration that readily equilibrates (e.g., simultaneously evens) the tension in the lower lace 42 as the lower lacing closure tab 44 is extended from the ankle-brace boot 12. From the distal end of the ankle brace 10, the lower lace 42 passes from its lower distal fixation 43d at the interior surface of the ankle support sleeve's first side-sleeve section 22 to and through an optional lower first outer distal eyelet 47a as depicted in FIG. 1.

With respect to FIGS. 1-4 as viewed from the patient's perspective, the ankle support sleeve's first side-sleeve section 22 is to the patient's left and the ankle support sleeve's second side-sleeve section 24 is to the patient's right. In one alternative embodiment (not shown), the distal end portion of the lower lace 42 can be fixed to the tongue 30 or another part of the ankle-brace boot 12 (e.g., an eyelet). In another alternative embodiment (not shown), the lower lace 42 is not immovably affixed to the ankle support sleeve 20 but rather knotted or otherwise configured (e.g., enlarged) so that the distal end portion of the lower lace 42 cannot pass through the lower first outer distal eyelet 47a or the like. For example, an end portion of the lower lace 42 can be doubled over, looped, or otherwise provided with any other suitable kind of obstruction, such as an enlargement (e.g., formed by deforming or melting), a whipping knot, a T-bar, or the like. In yet another alternative embodiment (not shown), the lower lace 42 can be fixed (e.g., stitched) to the exterior surface of the ankle support sleeve's first side-sleeve section 22, thereby rendering unnecessary the lower first outer distal eyelet 47a.

As illustrated in the exemplary ankle-brace embodiment depicted in FIG. 1, from the lower first outer distal eyelet 47a, the lower lace 42 passes to and through a lower second outer distal eyelet 49b, to and through a lower distal closure-tab eyelet 45c, to and through a lower second inner distal eyelet 49d, and to and through a lower first inner distal eyelet 47e. The lower lace 42 then passes lengthwise along the interior surface of the ankle support sleeve's first side-sleeve section 22 (e.g., along the interior surface of the first outer side-sleeve strip 23) from the lower first inner distal eyelet 47e to and through a lower first inner proximal eyelet 47f so that, between the lower first inner distal eyelet 47e and the lower first inner proximal eyelet 47f, the lower lace 42 is substantially parallel to the adjacent outer edge of the ankle support sleeve 20. The lower lace 42 then passes from the lower first inner proximal eyelet 47f to and through a lower second inner proximal eyelet 49g, to and through a lower proximal closure-tab eyelet 45h, to and through a lower second outer proximal eyelet 49i, to and through an optional lower first outer proximal eyelet 47j, and to a lower proximal fixation 43p at the interior surface of the ankle support sleeve's first side-sleeve section 22.

In one alternative embodiment (not shown), the proximal end portion of the lower lace 42 can be fixed to the tongue 30 or another part of the ankle-brace boot 12 (e.g., an eyelet). In another alternative embodiment (not shown), the lower lace 42 is not immovably affixed to the ankle support sleeve 20 but rather knotted or otherwise configured (e.g., enlarged) so that the proximal end portion of the lower lace 42 cannot pass through the lower first outer proximal eyelet 47j or the like. For example, an end portion of the lower lace 42 can be doubled over, looped, or otherwise provided with any other suitable kind of obstruction, such as an enlargement (e.g., formed by deforming or melting), a whipping knot, a T-bar, or the like. In yet another alternative embodiment (not shown), the lower lace 42 can be fixed (e.g., stitched) to the exterior surface of the ankle support sleeve's first side-sleeve section 22, thereby rendering unnecessary the lower first outer proximal eyelet 47j.

The exemplary ankle-brace embodiment depicted in FIGS. 1-4 also includes a reciprocal, upper lacing interconnection system 50. Whereas the lower lacing interconnection system 40 closes from right to left from the patient's perspective, the upper lacing interconnection system 50 closes from left to right.

The exemplary upper lacing interconnection system 50 shown in FIGS. 1-4 includes an upper lace 52 (e.g., exactly one lace) that moveably connects an upper lacing closure tab 54 (via one or more lace-redirection mechanisms, such as upper closure tab eyelets 55) to upper lace-redirection mechanisms (e.g., upper eyelets 57, 59) positioned on the ankle support sleeve 20. Those having ordinary skill in the art will appreciate that the upper lacing closure tab 54 is moveably connected to the upper lace 52. When the upper lacing closure tab 54 is not releasably secured to another part of the ankle brace (e.g., releasably affixed to the ankle support sleeve 20), the upper lacing closure tab 54 can move substantially freely along the upper lace 52 (e.g., the upper lace 52 can pass through the upper closure tab eyelets 55).

With reference to the exemplary ankle-brace embodiment depicted in FIGS. 1-4, four upper first side-sleeve eyelets 57 are formed through or otherwise positioned upon the ankle support sleeve's first side-sleeve section 22 near its free front edge (e.g., four upper eyelets are positioned in the first outer side-sleeve strip 23), and upper second side-sleeve eyelets 59 are formed through or otherwise positioned upon the ankle support sleeve's second side-sleeve section 24 near its free front edge (e.g., four upper eyelets are positioned in the second outer side-sleeve strip 25).

The upper lacing closure tab 54 includes a releasable-attachment mechanism, such as a hook-and-loop surface, to releasably secure the upper lacing closure tab 54 to another part of the ankle brace 10. As illustrated in FIGS. 1-4, two upper closure-tab eyelets 55c, 55h are formed through or otherwise positioned upon the upper lacing closure tab 54. The upper closure-tab eyelets 55 help to ensure that the upper lacing closure tab 54 remains centered on the ankle support sleeve 20 as the upper lacing closure tab 54 is pulled over and around the ankle-brace boot 12.

The upper lace 52 is freely threaded (or otherwise interlaced) though the upper first side-sleeve eyelets 57, the upper second side-sleeve eyelets 59, and the upper closure-tab eyelets 55, respectively. The upper lace 52 can move freely to accommodate the typical non-cylindrical anatomy of a patient's lower leg in which the leg tapers from the calf to the ankle (e.g., a frustoconical shape). This free movement of the upper lace 52 facilitates consistent tension along its length. This even tension is transferred to the upper first side-sleeve eyelets 57, the upper second side-sleeve eyelets 59, and the upper closure-tab eyelets 55 through which the upper lace 52 is interlaced. The upper lace 52 and the moveably attached upper lacing closure tab 54 wrap circumferentially around—rather than helically along—the patient's lower leg in a way that uniformly closes and tensions the ankle-brace boot 12.

Typically, an upper lace 52 has (i) a first end portion secured (e.g., affixed) to the ankle support sleeve 20 approximately midway along the ankle support sleeve 20 (e.g., secured midway along the ankle support sleeve 20 toward the distal end of the first outer side-sleeve strip 23 or the distal end of the second outer side-sleeve strip 25) and (ii) a second end portion secured (e.g., affixed) to the ankle support sleeve 20 near the ankle support sleeve's proximal end (e.g., secured at either the proximal end of the first outer side-sleeve strip 23 or the proximal end of the second outer side-sleeve strip 25).

In the exemplary ankle-brace embodiment depicted in FIGS. 1-4, one upper lace 52 has (i) a first distal end fixed to the ankle support sleeve's interior surface nearer the ankle support sleeve's distal end (i.e., an upper distal fixation 53d secured midway along the ankle support sleeve 20 toward the toe-side end of the second outer side-sleeve strip 25) and (ii) a second proximal end fixed to the ankle support sleeve's interior surface nearer the ankle support sleeve's proximal end (i.e., an upper proximal fixation 53p toward the knee-side end of the second outer side-sleeve strip 25). In the exemplary ankle-brace embodiment depicted in FIGS. 1-4, four upper second side-sleeve eyelets 59a, 59e, 59f, 59j are positioned at the ankle support sleeve's second side-sleeve section 24 between the upper lace's upper distal fixation 53d and the upper lace's upper proximal fixation 53p, and four upper first side-sleeve eyelets 57b, 57d, 57g, 57i are positioned at the ankle support sleeve's first side-sleeve section 22. As noted, the first outer side-sleeve strip 23 and the second outer side-sleeve strip 25 can function as eyestays (e.g., the four upper second side-sleeve eyelets 59a, 59e, 59f, 59j can be formed through second outer side-sleeve strip 25, and the four upper first side-sleeve eyelets 57b, 57d, 57g, 57i can be formed through the first outer side-sleeve strip 23).

As illustrated in FIGS. 1-4 in which the ends of upper lace 52 are fixed to the interior surface of the second side-sleeve section 24 (e.g., at the second outer side-sleeve strip 25 on the right side as viewed from the patient's perspective), pulling the upper lacing closure tab 54 away from the ankle support sleeve's first side-sleeve section 22 (and over and around the ankle support sleeve's second side-sleeve section 24) uniformly closes and tensions the ankle-brace boot 12 by drawing together the ankle support sleeve's first side-sleeve section 22 and ankle support sleeve's second side-sleeve section 24. As viewed from the patient's perspective, the upper lacing interconnection system 50 closes from left to right as shown in the exemplary ankle brace of FIGS. 1-4.

As illustrated in the exemplary ankle-brace embodiment depicted in FIGS. 1-4, the ankle brace 10 includes a compression-enhancing lacing configuration that readily equilibrates (e.g., simultaneously evens) the tension in the upper lace 52 as the upper lacing closure tab 54 is extended from the ankle-brace boot 12. Near the middle of the ankle brace 10, the upper lace 52 passes from its upper distal fixation 53d at the interior surface of the ankle support sleeve's second side-sleeve section 24 to and through an optional upper second outer distal eyelet 59a as depicted in FIG. 2.

With respect to FIGS. 1-4 as viewed from the patient's perspective, the ankle support sleeve's second side-sleeve section 24 is to the patient's right and the ankle support sleeve's first side-sleeve section 22 is to the patient's left. In one alternative embodiment (not shown), the distal end portion of the upper lace 52 can be fixed to the tongue 30 or another part of the ankle-brace boot 12 (e.g., an eyelet). In another alternative embodiment (not shown), the upper lace 52 is not immovably affixed to the ankle support sleeve 20 but rather knotted or otherwise configured (e.g., enlarged) so that the distal end portion of the upper lace 52 cannot pass through the upper second outer distal eyelet 59a or the like. For example, an end portion of the upper lace 52 can be doubled over, looped, or otherwise provided with any other suitable kind of obstruction, such as an enlargement (e.g., formed by deforming or melting), a whipping knot, a T-bar, or the like. In yet another alternative embodiment (not shown), the upper lace 52 can be fixed (e.g., stitched) to the exterior surface of the ankle support sleeve's second side-sleeve section 22, thereby rendering unnecessary the upper second outer distal eyelet 59a.

As illustrated in the exemplary ankle-brace embodiment depicted in FIG. 2, from the upper second outer distal eyelet 59a, the upper lace 52 passes to and through a upper first outer distal eyelet 57b, to and through an upper distal closure-tab eyelet 55c, to and through an upper first inner distal eyelet 57d, and to and through an upper second inner distal eyelet 59e. The upper lace 52 then passes lengthwise along the interior surface of the ankle support sleeve's second side-sleeve section 24 (e.g., along the interior surface of the second outer side-sleeve strip 25) from the upper second inner distal eyelet 59e to and through a upper second inner proximal eyelet 59f so that, between the upper second inner distal eyelet 59e and the upper second inner proximal eyelet 59f, the upper lace 52 is substantially parallel to the adjacent outer edge of the ankle support sleeve 20. The upper lace 52 then passes from the upper second inner proximal eyelet 59f to and through an upper first inner proximal eyelet 57g, to and through an upper proximal closure-tab eyelet 55h, to and through an upper first outer proximal eyelet 57i, to and through an optional upper second outer proximal eyelet 59j, and to an upper proximal fixation 53p at the interior surface of the ankle support sleeve's second side-sleeve section 24.

In one alternative embodiment (not shown), the proximal end portion of the upper lace 52 can be fixed to the tongue 30 or another part of the ankle-brace boot 12 (e.g., an eyelet). In another alternative embodiment (not shown), the upper lace 52 is not immovably affixed to the ankle support sleeve 20 but rather knotted or otherwise configured (e.g., enlarged) so that the proximal end portion of the upper lace 52 cannot pass through the upper second outer proximal eyelet 59j or the like. For example, an end portion of the upper lace 52 can be doubled over, looped, or otherwise provided with any other suitable kind of obstruction, such as an enlargement (e.g., formed by deforming or melting), a whipping knot, a T-bar, or the like. In yet another alternative embodiment (not shown), the upper lace 52 can be fixed (e.g., stitched) to the exterior surface of the ankle support sleeve's second side-sleeve section 24, thereby rendering unnecessary the upper second outer proximal eyelet 59j.

In ankle-brace embodiments in which an end portion of a lace is fixedly connected to an eyelet or otherwise restricted with respect to an eyelet (e.g., by knotting to restrict the end of the lace from passing through the eyelet), the eyelet may function as a mount rather than a lace-redirection mechanism.

The exemplary ankle braces depicted in FIGS. 1-6 include both a lower lacing interconnection system 40 and an upper lacing interconnection system 50. Neither of these lacing interconnection systems 40, 50 fixedly attaches its single lace 42, 52 to the corresponding lacing closure tab 44, 54, either fully or partially. Rather, in these exemplary ankle-brace embodiments, each lace 42, 52 may pass substantially freely through each of the corresponding closure-tab eyelets 45, 55. As depicted in FIGS. 1-6, each strand of lace 42, 52 connecting the ankle support sleeve 20 with a corresponding lacing closure tab 44, 54 is freely threaded through a corresponding closure-tab eyelet 45, 55. This “free-floating” closure-tab design enables the patient to readily equilibrate lace tension through the various eyelets positioned along the length of the ankle support sleeve 20 in a way that applies substantially consistent compression to the patient's lower leg and ankle. In this regard, each lace 42, 52 and its corresponding, moveably attached lacing closure tab 44, 54 wrap circumferentially around—rather than helically along—the patient's lower leg and ankle in a way that uniformly closes and tensions the ankle-brace boot 12.

In exemplary ankle-brace embodiments such as depicted in FIG. 2, the lower lace 42, the upper lace 52, or both pass through one or more lacing channels, which may be secured to or otherwise formed in the tongue 30. For example, one or more lacing channels 62 may be positioned near the midpoint, near the distal end, and/or near the proximal end of a cushioned, stretchable tongue 30. The lower lace 42 may pass through a lower lacing channel 62 secured to the tongue 30 as the lower lace 42 passes from the lower first outer distal eyelet 47a to the lower second outer distal eyelet 49b, or the lower lace 42 may pass through a lower lacing channel 62 secured to the tongue 30 as the lower lace 42 passes from the lower second outer proximal eyelet 49i to the lower first outer proximal eyelet 47j. Similarly, the upper lace 52 may pass through an upper lacing channel 62 secured to the tongue 30 as the upper lace 52 passes from the upper second outer distal eyelet 59a to the upper first outer distal eyelet 57b (FIG. 2), or the upper lace 52 may pass through an upper lacing channel 62 secured to the tongue 30 as the upper lace 52 passes from the upper first outer proximal eyelet 57i to the upper second outer proximal eyelet 59j.

As will be appreciated by those having ordinary skill in the art, each lacing channel 62 should be positioned to facilitate passage of either the lower lace 42 or the upper lace 52 between the ankle support sleeve's first side-sleeve section 22 and the ankle support sleeve's second side-sleeve section 24 (e.g., between the first outer side-sleeve strip 23 and second outer side-sleeve strip 25). Positioning lacing channel(s) 62 at either the distal end or the proximal end of the tongue 30, or both the distal end and the proximal end of the tongue 30, helps to maintain the proper positioning of the tongue 30 against the patient's lower leg. In practice, securing a lace 42, 52 to the tongue 30 via one or more lacing channels 62 (e.g., at the distal end and/or the proximal end of a cushioned, stretchable tongue 30) reduces patient discomfort by preventing the lace 42, 52 from migrating beyond the end of the tongue 30 when the ankle brace 10 is compressively and securely applied to the patient's ankle and lower leg.

As illustrated in the exemplary ankle-brace embodiments depicted in FIGS. 1-4 and FIGS. 5-6, hook-and-loop fasteners 65 (e.g., VELCRO-brand fasteners) are secured to the surfaces of the ankle brace 10 to facilitate closure of the ankle brace 10 to the patient's lower leg, ankle, and foot. For example, the ankle support sleeve 20, including the first side-sleeve section 22 and the second side-sleeve section 24, include a surficial fabric that functions as a loop fastener. Alternatively, loop-fasteners 66 (e.g., loop-fastener strips) can be secured to the outer surfaces of the ankle support sleeve 20, typically at the ankle support sleeve's first side-sleeve section 22 and the ankle support sleeve's second side-sleeve section 24. Hook-fasteners 67 are provided elsewhere on the ankle brace 10, such as on a surface of each lacing closure tab 44, 54.

As illustrated in FIGS. 1-6, a patient may secure the ankle brace 10 to his ankle via one or more lacing closure mechanisms (e.g., the lower lacing interconnection system 40 and the upper lacing interconnection system 50). Wrapping the respective lacing closure tabs 44, 54 circumferentially around the ankle-brace boot 12 evenly closes and tensions the ankle-brace boot 12 by drawing together the ankle support sleeve's first side-sleeve section 22 and the ankle support sleeve's second side-sleeve section 24. Depending on the girth of the patient's lower leg, the hook-fasteners 67 on a surface of each lacing closure tab 44, 54 will engage loop-fasteners 66 positioned upon the respective outer surfaces of ankle support sleeve 20.

The advantageous lacing interconnection systems according to the present invention may be incorporated in other kinds of medical support braces, such as those having alternative ankle-brace designs. For example, the exemplary ankle-brace embodiment depicted in FIGS. 5-6 includes stabilizing straps 71 and optional binding straps 72, similar to those disclosed in commonly assigned U.S. Pat. No. 5,067,486 for an Ankle Stabilizing Appliance (Hely). Likewise, the present lacing interconnection systems can enhance the ankle braces disclosed in commonly assigned U.S. Pat. No. 8,721,578 for an Ankle Stabilizing Apparatus Having a Dynamic Cuff and Stabilizing Strap System (Gaylord) and commonly assigned U.S. Pat. No. 9,737,430 for an Ankle Stabilizing Apparatus Having a Dynamic Cuff and Stabilizing Strap System (Gaylord).

Therefore, to supplement the present disclosure, this application incorporates entirely by reference the following commonly assigned patents and patent application publications: U.S. Pat. No. 5,067,486; U.S. Pat. No. 5,795,316; U.S. Pat. No. 7,651,472; U.S. Pat. No. 8,721,578; U.S. Pat. No. 8,808,215; U.S. Pat. No. 9,375,339; U.S. Pat. No. 9,393,146; U.S. Pat. No. 9,737,430; U.S. Patent Application Publication No. 2009/0112140 A1; U.S. Patent Application Publication No. 2014/0249460 A1; U.S. Patent Application Publication No. US2017/0143526 A1; and U.S. Patent Application Publication No. US2017/0143530 A1.

In the specification and/or figures, typical embodiments of the ankle-brace invention have been disclosed. The present ankle-brace invention is not limited to such exemplary embodiments. The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

1. An ankle brace, comprising:
an ankle support sleeve having a first side-sleeve section and a second side-sleeve section;
first side-sleeve lace-redirection mechanisms connected to the ankle support sleeve's first side-sleeve section;
second side-sleeve lace-redirection mechanisms connected to the ankle support sleeve's second side-sleeve section;
a lacing closure tab having one or more closure-tab lace-redirection mechanisms; and
a lace having (i) a first end portion secured to the ankle support sleeve nearer the ankle support sleeve's distal end and (ii) a second end portion secured to the ankle support sleeve nearer the ankle support sleeve's proximal end, wherein the lace is freely threaded though the respective first side-sleeve lace-redirection mechanisms, second side-sleeve lace-redirection mechanisms, and the one or more closure-tab lace-redirection mechanisms.
